(19) United States
(12) Patent Application Publication
(10) Pub. No.: US 2022/0304832 A1
(43) Pub. Date: Sep. 29, 2022

(54) ORTHOPAEDIC DEVICE AND ENERGY STORAGE DEVICE

(71) Applicant: Ottobock SE & Co. KGaA, Duderstadt (DE)

(72) Inventors: Christian Will, Gottingen (DE); Torsten Parth, Engelsbach (DE); Leonard Vier, Norten-Hardenberg (DE); Carsten Moenicke, Duderstadt (DE); Georg Gehrmann, Gottingen (DE); Andreas Bohland, Wien (AT); Jessica Gabriela Beltran Ullauri, Herzberg (DE); Viktor Gerhard Horig, Herzberg (DE); Herman Boiten, Ede (NL)

(21) Appl. No.: 17/635,751

(22) PCT Filed: Aug. 17, 2020
(86) PCT No.: PCT/EP2020/072968

§ 371 Date: Feb. 16, 2022

(30) Foreign Application Priority Data
Aug. 20, 2019 (DE) 10 2019 122 372.5

(51) Int. Cl. A61F 2/64; A61F 2/74; A61F 2/70

(52) U.S. Cl. CPC A61F 2/644; A61F 2/748; A61F 2/70; A61F 2/744; A61F 2002/5006

(57) ABSTRACT

The invention relates to an orthopedic device with an energy storage device 2 that comprises at least one cylinder 4, in which a first cylinder chamber 6, a second cylinder chamber 8, which is fluidically connected to the first cylinder chamber 6 by at least one fluid line 14, and a piston 10 are located, wherein the piston 10 is arranged relative to the cylinder 4 such that it can be displaced in such a way that by displacing the piston 4, an operating medium, which is a fluid, is conveyed through the at least one fluid line 14 from one cylinder chamber 6, 8 into the other cylinder chamber 8, 6, wherein the energy storage device 2 has at least one compensation volume 24, which is fluidically connected to the fluid line 14 via a fluid connection 22, and a first controllable valve 26, by means of which the fluid connection 22 can be opened and closed.

### CROSS-REFERENCE TO RELATED APPLICATION(S)

This is a national stage application filed under 37 U.S.C. 371 based on International Patent Application No. PCT/EP2020/072968, filed Aug. 17, 2020, which application claims priority to German Patent Application No. 10 2019 122 372.5 filed with the German Patent Application Office on Aug. 20, 2019, the disclosure of which is incorporated herein by reference in its entirety.

The invention relates to an orthopedic device with an energy storage device that has at least one cylinder, in which a first cylinder chamber, a second cylinder chamber, which is fluidically connected to the first cylinder chamber by at least one fluid line, and a piston are located, the piston being arranged relative to the cylinder such that it can be displaced and such that the displacement of the piston causes an operating medium, which is a fluid, to be conveyed through the at least one fluid line from one cylinder chamber into the other cylinder chamber. The invention also relates to an energy storage device for such an orthopedic device.

Orthopedic devices in many forms have been known within the scope of the prior art for many years. This includes, for example, prostheses, especially knee prostheses, ankle prostheses, foot prostheses, elbow prostheses or hand prostheses.

Orthopedic devices also include orthoses, particularly knee orthoses, ankle orthoses, foot orthoses, elbow orthoses or hand orthoses.

Exoskeletons which are arranged externally on a part of the body or the entire body of the wearer and are intended to enable movements and/or activities which can no longer be performed by the body itself are also orthopedic devices within the meaning of this invention. It also includes devices which make it easier for the wearer to perform energy-intensive, strenuous or tiring activities, such as overhead work, better, easier, faster and longer.

Many of these orthopedic devices have joints which are intended to replace or support, sustain or protect joints of the wearer of the orthopedic device which are no longer present. In many cases, it is desirable and advantageous if the orthopedic device absorbs energy during a movement of one of its joints, for example, temporarily stores this energy and releases it again at a later point, for example in a step cycle. This is desirable with knee prostheses and knee orthoses, for example, in which energy is stored in an energy storage device, preferably when the knee is bent, and is released again when the knee is extended.

A number of energy storage devices are known from the prior art which are usually designed as spring elements, such as hydraulic or mechanical spring elements. If the joint is bent, the spring element is compressed and charged with energy. This is released again by the spring element at a later point. However, it is a disadvantage that this release of energy occurs immediately after the force responsible for the energy charge disappears and is uncontrolled and instantaneous. A temporary storage of energy or controlled release of the stored energy is not or is only barely possible with such simple systems.

Damping elements or resistors are therefore known from the prior art, by which a movement of, for example, the joint of the orthopedic device is rendered more difficult or delayed. This is the case with hydraulic systems, for example, in which a fluid acting as an operating fluid is moved between two cylinder chambers, for example, when the piston of the hydraulic device is moved. If there is a throttle valve in the fluid line between the two cylinder chambers, the flow resistance opposing the flowing fluid can be adjusted with this throttle valve. If the valve is completely closed, no fluid can flow and the joint of the orthopedic device is blocked.

However, it is a disadvantage that such systems do not allow for the energy to be stored, so that only a passive resistance is achieved.

If both effects, i.e. energy storage and controlled release, are desired, both types of system are usually combined with each other. For example, corresponding systems are known from U.S. Pat. No. 9,416,838 B2 and WO 2016/171548. However, since these are actually combinations of two systems, the energy storage devices are structurally complex and therefore prone to error and expensive.

Furthermore, with all of these systems it is barely possible to enable free movement of the joint of the orthopedic device.

The invention therefore aims to further develop an energy storage device in such a way that it can be used flexibly and can be manufactured in a more space-saving and structurally simpler manner.

The invention solves the problem by way of an orthopedic device according to the preamble of claim 1, which is characterized in that the energy storage device comprises at least one compensation volume, which is fluidically connected to the fluid line by a fluid connection, and at least one controllable valve, by means of which the fluid connection can be opened and closed.

If the fluid connection is closed by the first controllable valve, a fluid exchange between the compensation volume and the remaining components, in particular the first cylinder chamber or the second cylinder chamber, cannot take place. If in this state the piston is moved, the operating medium is pushed from one of the two cylinder chambers through the fluid line into the respective other cylinder chamber. The flow resistance caused by the fluid line counteracts the operating medium, so that stronger or less strong damping is caused depending on the size of the flow resistance.

In a preferred embodiment, when the piston is displaced inside the cylinder when the first controllable valve is closed, the overall volume that is available for the operating medium, i.e. the fluid, changes. Conventionally, the piston is fixed to a piston rod, for example, and is displaced on this rod inside the cylinder. This means that the volume of the cylinder chamber in which the piston rod is situated is reduced by the piston rod. If the piston is consequently displaced in such a way that a larger section of the piston rod is arranged in the respective cylinder chamber, the overall volume available for the operating medium is reduced, so that the pressure is increased. Depending on the compressibility of the operating medium, it is thus possible to at least almost, preferably even completely, prevent movement, for example if the fluid that forms the operating medium is incompressible. If the operating medium is not completely incompressible, the resistance that counters the displacement of the piston is intensified as displacement increases. The operating medium inside the cylinder chambers and the fluid line is thus compressed, thereby acting as an energy store. Within the scope of the present invention, an operating medium is preferably considered to be completely incompressible if the forces occurring during the intended use of the orthopedic device do not lead to a displacement of the piston when the fluid connection is closed.

In this embodiment, when the first controllable valve is closed, the energy storage device acts as a spring element. The spring constant depends largely on the compression modulus of the operating medium used. If the system is loaded, for example a force is applied to the piston that acts in the direction of the first cylinder chamber, the piston is displaced in this direction. As a result, in this embodiment the section of the piston rod inside the cylinder increases, so that, as already explained above, the volume available for the operating medium decreases. The pressure on the operating medium therefore increases and the displacement of the piston ends when the pressure of the operating medium offsets the force acting externally on the piston.

How large the spring deflection, i.e. the displacement of the piston, is for a given external force therefore depends on how large the change in volume of the volume is in relation to the total volume available to the operating medium. This change in volume depends on the diameter of the piston rod that causes the change in volume.

The smaller the diameter of the piston rod, the lower the hardness of the spring and therefore the greater the spring deflection. To obtain a soft spring, it is therefore advantageous to use a particularly thin piston rod, i.e. one with a small diameter. However, below a critical diameter, this is at the expense of the mechanical stability of the piston rod. The spring constant preferably also depends on the volume of the first cylinder chamber and the volume of the second cylinder chamber. The spring constant can also be formulated as being dependent on the ratio of the two volumes. Consequently, the spring constant can be reduced for a given diameter of the piston rod, i.e. the spring deflection can be increased for a given force by increasing the overall volume available to the operating medium. However, this generally causes an increase in the size of the energy storage device, meaning it requires more installation space.

In a preferred embodiment, the piston rod extends into, especially preferably through, both cylinder chambers, but has different diameters or cross-sectional surfaces in the two cylinder chambers. Only the difference contributes to the change in volume, so that even small changes in volume and thus soft springs can be realized.

A piston rod has a diameter of less than 10 mm, for example, especially preferably less than 7 mm.

A wall of the cylinder is preferably designed in such a way that it acts as a mechanical energy store. This can be achieved, for example, by an area with a very low wall thickness that deforms elastically under the influence of corresponding pressures.

The storage of energy can be prevented by using the first controllable valve to open the fluid connection between the compensation volume and the remaining elements of the hydraulic system. In this case, when the piston is displaced inside the cylinder, the overall volume of the two cylinder chambers still changes, but it can be offset by the compensation volume, so that the operating medium is not compressed and therefore no energy is stored. A damping of the movement of the piston still occurs, as the flow resistance of the fluid line still counters the transport of the operating medium. If the overall volume available inside the two cylinder chambers decreases when the piston is displaced inside the cylinder, part of the operating medium is pushed through the fluid line and the fluid connection into the compensation volume. When the piston moves in the opposite direction inside the cylinder, the operating medium is suctioned out of the compensation volume again, so that the original state is restored. In this way, the energy storage device can be used as a damping energy store or it enables a movement of the piston without a temporary storage of energy.

If the first controllable valve is open and the compensation volume is therefore not disconnected from the rest of the system, a temperature equalization may also occur. Here, a change in volume of the operating medium caused by changes in temperature is offset by the compensation volume. It is thus possible to prevent the spring properties from changing with the temperature.

In a preferred embodiment of the invention, the operating medium is a compressible fluid, preferably an oil, especially preferably a silicone oil. It is also advantageous that the stored energy that is stored when the fluid is compressed is almost completely released again when the load is removed and the oil can be used in a space-saving manner due to its fluidity, which enables it to effectively fill the form, and it can absorb high forces.

Such an operating medium blurs the boundaries between a hydraulic system, which uses a liquid as the operating medium, generally assumed to be incompressible, and a pneumatic system, which uses a gas as the operating medium. This is generally compressible.

In orthopedic devices of the prior art, the compressibility of the fluids is not used in the hydraulic arrangements. Rather, operating mediums are usually used that are considered to be incompressible and are used as such. Typically in such hydraulics, controllable valves are provided between the two cylinder chambers in order to achieve and control a damping of the movement. In addition, a permanently connected compensation volume can be provided.

Especially when using knee joints, high pressures of up to 200 bar are generated by the forces that occur, which act on the operating medium of the hydraulic arrangement of the knee joint. With typical volumes of 25 ml hydraulic oil and a piston diameter of 25 mm and a piston rod diameter of 10 mm, this means that with conventional hydraulic oil and closed valves there is only a maximum piston path of approx. 0.4 mm, which is too little to obtain a noticeable spring effect or to store a relevant amount of energy over a usable piston path. Within the meaning of the present invention, such a hydraulic oil in this hydraulic arrangement is deemed completely incompressible. In this case, blocking the valves therefore corresponds to a fully blocked joint with no energy storage function. In this case, opening the valves therefore corresponds to a completely free joint with no energy storage function.

Damping behavior can be regulated via the valve position, such that a controllable valve in front of the compensation volume is neither necessary nor provided.

Conversely, in the device according to the invention, the compressibility of the fluid is used for storing energy. To this end, the ratio between compression modulus of the fluid, piston rod diameter and volume of the cylinder chambers is selected in such a way that the desired energies can be stored without generating excessively high pressures or the piston's retraction path becoming too short.

An example of a requirement of a knee joint is that it stores enough energy to support the user when they stand up. The compensation volume must now feature a controllable valve to be able to switch between the energy storage function and the damping function. When the compensation volume is switched on, the hydraulics behave as described above; blocking and damping can be achieved via at least one controllable valve in the fluid line. When the fluid connection is closed, the compensation volume is fluidically decoupled from the rest of the system. If any available valves in the fluid line are opened or such a valve is not available, energy can be stored in the system, but free movement is no longer possible.

The operating medium used as a fluid preferably has a compression modulus of less than 1.5 GPa, especially preferably less than 1.2 GPa. It is therefore possible to select the remaining parameters, i.e. especially pressure, volume of the respective cylinder chambers and piston rod diameter, to lie within a range that is technically more feasible and in particular to construct a smaller device, approximately on a scale that is acceptable for orthopedic devices. In the knee joint given as an example above with conventional hydraulics and 25 ml operating fluid, a longer piston path can be achieved with the same maximum pressure of 200 bar, for example, by using silicone oil with a compression modulus of 1.5 GPa instead of the conventional hydraulic oil and reducing the diameter of the piston rod to 6 mm. If the fluid connection is closed, i.e. the compensation volume is decoupled and the second controllable valve is open, this results in a piston path of 7 mm up to the maximum 200 bar. When using an operating medium with a compression modulus of 1 GPa, even 10.5 mm can be achieved.

Via the selection of piston rod diameter, volume of the cylinder chambers and compression modulus, different spring constants, i.e. stiffnesses, can be set. In the stance phase of the gait cycle, a natural stiffness of the knee is preferably reproduced, which corresponds to a linear spring constant in a range between 0 N/mm to 750 n/mm. Preferably, a spring constant of less than 600 N/mm, especially preferably less than 400 N/mm, and preferably greater than 100 N/mm, especially preferably greater than 300 N/mm is set. With an exemplary spring constant of 400 N/mm, a force of 4320 N acts at a deflection angle of 25°. In this state, a potential energy of approximately 23 joules is stored. Furthermore, a path of 10.8 mm is achieved with the acting force when the compensation volume is completely decoupled.

The operating medium is preferably a magnetorheological fluid. These fluids have a viscosity or flow capacity that can be influenced by the effect of magnetic fields. In these embodiments, throttle valves and/or controllable valves can be designed as magnets, such as electromagnets. This renders expensive and complex mechanical components, as required for conventional mechanical valves, unnecessary. A line through which the operating medium flows, for example the fluid line, is arranged in such a way that a magnetic field of a magnet can influence it. If the magnetic field is increased, the flow capacity of the operating medium in the form of a magnetorheological fluid decreases, for example. This increases the flow resistance. Conversely, the flow resistance is reduced by weakening the magnetic field, as this causes an increase in the flow capacity of such an operating medium.

Preferably, at least a second controllable valve is located in the fluid line that connects the two cylinder chambers to one another, by way of which a flow resistance of the fluid connection can be adjusted, preferably infinitely. The fluid connection can preferably be completely closed by way of the second controllable valve. The second controllable valve is preferably a throttle valve. Alternatively, a throttle valve is also provided for adjusting the flow resistance. In this way, a damping of the movement of the piston inside the cylinder can be adjusted.

It is especially preferably for at least two second controllable valves, preferably two throttle valves, to be provided in the fluid line, between which the connection to the fluid connection is located. This ensures that the operating medium, i.e. the fluid, is always conveyed through one of the throttle valves when it is conveyed into or out of the compensation volume.

By way of the two second controllable valves, preferably the two throttle valves, the different flow resistances for the two directions of movement of the piston inside the cylinder and therefore different damping properties can be adjusted.

Preferably, at least one, but preferably each throttle valve, is bypassed by a non-return valve which allows a flow of the operating medium into the respective cylinder chamber, but prevents it from leaving this cylinder chamber.

In a preferred embodiment, the energy storage device features at least one additional volume that is fluidically connected to the first cylinder chamber, the energy storage device preferably having a third controllable valve by means of which the connection can be opened and closed. It is particularly preferable if the additional volume is fluidically connected to the at least one fluid line that connects the two cylinder chambers to each other.

This additional volume opens up further possibilities for using the energy storage device.

A compensation volume is able to hold operating medium without increasing the pressure on the operating medium. As previously explained, this renders it possible to offset the change in volume of the two cylinder chambers, which may occur when the piston is displaced. Consequently, a pressure equalization takes place. Conversely, such pressure equalization is not possible with an additional volume. It is therefore a closed volume, preferably completely filled with operating medium. With an energy storage device that features at least one of these additional volumes, the pressure of the medium in the additional volume increases or decreases when the piston is displaced inside the cylinder whenever the third controllable valve, which controls the connection to the compensation volume, is closed. For example, if the piston is displaced inside the cylinder in such a way that the volume of the two cylinder chambers available for the operating medium is reduced and at the same time the first controllable valve, which can open and close the fluid connection, is in the closed position, the pressure inside the operating medium not only increases inside the cylinder chambers, but also inside the additional volume.

In this state, the third controllable valve, which is responsible for connecting the additional volume to the hydraulic system, can be closed, so that the operating medium is stored inside the additional volume at a higher pressure, and therefore with more potential energy. The additional volume consequently serves as a sealable energy store, in which received energy can be stored by closing the respective third controllable valve. Irrespective of the position and/or movement of the piston inside the cylinder, the corresponding third controllable valve can be re-opened at any desired time in order to expand the operating medium inside the additional volume and release the potential energy stored within it.

In the case of a knee joint, the energy can be stored while sitting down, for example. The third controllable valve can subsequently be closed and the at least one second controllable valve in the fluid line, the first controllable valve and therefore the fluid connection opened. This results in the loss of the energy stored in the cylinder chambers, but the joint can be moved freely while sitting. Upon standing up, the at least first controllable valve in the fluid connection can be closed again and the third controllable valve opened, so that the energy stored in the additional volume can be used as support for standing up.

Furthermore, a change in stiffness can occur via the additional volume by opening or closing the third controllable valve. If the third controllable valve is opened, an overall grater volume of operating medium is available, which causes stiffness to decrease. By closing the third controllable valve, the overall volume available to the operating medium decreases and stiffness increases. This can be utilized for adjusting the stiffness depending on the situation: for example with a knee joint, a greater stiffness is required in the stance phase than when sitting down. Adapting the stiffness to the user of the orthopedic device may also be practical, for example depending on the user's weight or their personal preferences.

It is especially preferable for a throttle valve to be provided in this connection too, so that the flow resistance of the connection can be adjusted. This throttle valve can either be provided in addition to the third controllable valve or the third controllable valve is designed as a throttle valve. It is thus also possible to adjust how quickly the pressurized operating medium is expanded and over what period of time and at what speed the potential energy stored in it is released.

In a preferred embodiment, the energy storage device features multiple additional volumes. Preferably, they are all connected to the rest of the system, for example to one of the cylinder chambers. It is especially preferable if the energy storage device also has a plurality of third controllable valves, so that the connections of the individual additional volumes, preferably each individual additional volume, can be opened and closed separately. This is preferably done independently of each other. Different quantities of potential energy can therefore be stored in different additional volumes and released as necessary. The additional volumes may have the same volume or different volumes and be containers with different degrees of resistance to pressure. Multiple additional volumes also allow for a larger range of adjustable stiffnesses.

Several of these additional volumes are preferably fluidically connected to each other in series. This means that the volumes are “connected in series”. The part of the operating medium that is conveyed into the last of these additional volumes must consequently pass through all other additional volumes that are connected in series with this final additional volume.

Alternatively or additionally, several of the additional volumes are fluidically connected to each other in parallel. This means that the volumes are “connected in parallel”. This says nothing of the spatial orientation of the volumes. It only means that the operating medium to be conveyed into one of the additional volumes need not be conveyed through another of these parallel connected additional volumes.

In this case too, each individual additional volume can preferably be connected to or disconnected from the rest of the fluid system by a third controllable valve. It is especially preferable for a separate throttle valve to be provided for each additional volume, by means of which the flow resistances in the respective connection lines can be adjusted.

It is advantageous if the orthopedic device features at least one electric control unit that is configured to control the controllable valves, the switch valves and/or the throttle valves independently of each other. Such an electric control unit is an electronic data processing device, for example, that is configured to send control signals to the corresponding valves and thus bring the valves from one state into another. This may occur on the basis of sensor data, for example, determined by sensors, which may also form part of the orthopedic device. They can be force sensors, strain sensors, temperature sensors, speed or acceleration sensors, or other sensors.

The first piston is preferably mounted such that it can be displaced along a circular path, as is known from rotational hydraulics, for example.

The orthopedic device is preferably a knee prosthesis or a knee orthosis.

The invention also solves the problem by way of an energy storage device for one of the orthopedic devices described here.

In the following, examples of embodiments of the present invention will be explained in more detail by way of the attached drawings:

They show:

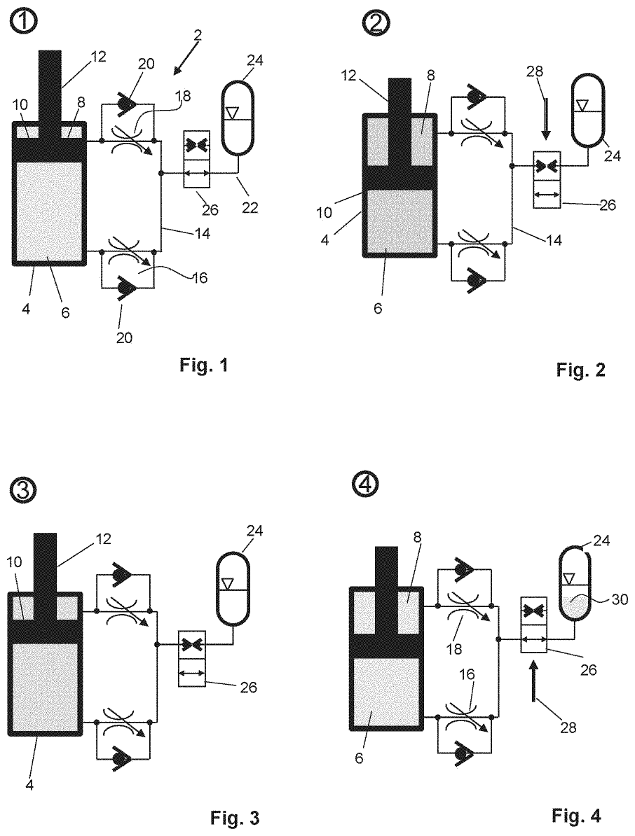

FIGS. 1 to 5-different states of an energy storage device according to a first example of an embodiment of the present invention, and

FIGS. 6 to 10-different states of a second embodiment of an energy storage device.

FIG. 1 schematically depicts an energy storage device 2 for an orthopedic device. The energy storage device features a cylinder 4, containing a first cylinder chamber 6 and a second cylinder chamber 8 that are separated from a piston 10, which is mounted in a piston rod 12.

The first cylinder chamber 6 is connected to the second cylinder chamber 8 via a fluid connection 14. In the fluid line 14 there is a first throttle valve 16 and a second throttle valve 18, each of which is bypassed by a non-return valve 20. The non-return valves 20 are arranged in such a way that no operating medium can escape the first cylinder chamber 6 when the first throttle valve 16 is closed and no operating medium can escape the second cylinder chamber 8 when the second throttle valve 18 is closed. In the example of an embodiment shown, the first throttle valve 16 with its assigned non-return valve 20 form a second controllable valve. The second throttle valve 18 and its assigned non-return valve 20 also form a second controllable valve.

Between the two throttle valves 16, 18, a compensation volume 24 is fluidically connected via a fluid connection 22 to the fluid line 14 and thus to the first cylinder chamber 6 and the second cylinder chamber 8. In the fluid connection 22 there is a first controllable valve 26 that can be brought into an open state, depicted in FIG. 1, and a closed state by disconnecting the compensation volume 24 from the rest of the fluid system. Such an energy storage device 2, as schematically depicted in FIGS. 1 to 5, may be arranged in a prosthetic knee, for example, so that a step cycle as described in FIGS. 1 to 5 can take place.

FIG. 1 shows the situation upon heel strike. The compensation volume 24 is connected to the fluid line 14 via the open switch valve 26. The first throttle valve 16 and the second throttle valve 18 are open, wherein openings of different sizes can be achieved by the respective throttle valves 16, 18, so that the flow resistance countering a fluid movement can be adjusted.

Upon heel strike, a flexion of the prosthetic knee occurs, the energy storage device 2 being installed in said prosthetic knee. As a result, the piston 10 is displaced downwards in the cylinder 4. This situation is depicted in FIG. 2. The piston 10 has been displaced downwards, thereby making the first cylinder chamber 6 smaller. At the same time, the second cylinder chamber 8 has been enlarged. However, the overall volume of the two cylinder chambers 6, 8 has decreased, as a larger part of the piston rod 12 is now arranged inside the cylinder 4. When the piston 10 was lowered, the situation shown in FIG. 1 prevailed so that the compensation volume 24 is connected to the rest of the fluid system. Since the overall volume of both cylinder chambers 6, 8 decreased while lowering the piston 10, part of the fluid was pressed into the compensation volume 24.

In FIG. 2, the arrow 28 indicates that the first controllable valve 26 is closed, for example, during the so-called “foot flat”, when the entire foot rests on the ground. As a result, the connection to the compensation volume 24 and the fluid line 14 is disconnected. The part of the operating medium that was pushed into the compensation volume 24 when the foot was lowered and thus the piston 10 was lowered inside the cylinder 4 can no longer leave this compensation volume 24. A further flexion of the prosthetic knee, in which the energy storage device 2 is installed, would cause the piston 10 to be lowered further and therefore to a further reduction in overall volume of the two cylinder chambers 6, 8. This would result in a compression of the fluid contained within, for example a silicone oil. As a result, the pressure inside the silicone oil is increased and thus potential energy stored. As there in no way for the operating medium to leave the system from the first cylinder chamber 6, the second chamber 8 and the fluid line 14, the energy is stored in this system and released again when the inflecting force decreases. In this way, for example, the natural stance phase flexion angles of up to 25° can now be achieved with a prosthetic knee without the user having to worry that the stored energy is lost so that they can no longer extend the knee independently from this flexion.

The energy storage device 2 stores the further supplied potential energy from the moment the switching valve 26 closes and then releases it again. This pushes the piston 10 in FIG. 2 upwards, as the pressure in the two cylinder chambers 6, 8 is identical, but the lower side of the piston 10 exposed to the pressure is greater than the upper side exposed to the pressure, so that an overall upward force is achieved.

This situation is depicted in FIG. 3. The piston 10 with the piston rod 12 has been pushed upwards. This occurs until the position in which the first controllable valve 26 was closed. If, unlike in FIG. 2, this already occurs at an earlier point in time, i.e. when a piston 10 with piston rod 12 has not been inserted so far into the cylinder 4, the position shown in FIG. 3 can be achieved. The switch valve 26 remains closed.

If, contrary to the figures shown, the switch valve 26 is closed immediately upon heel strike, i.e. in the position depicted in FIG. 1, there is no fluid inside the compensation volume 24, as the switch valve 26 was closed already before the volume of the two cylinder chambers 6, 8 was compressed for the first time.

The arrangement depicted renders it possible to release absorbed potential energy from the moment that the switch valve 26 is closed by bending the prosthetic knee or another joint of an orthopedic device, thereby supporting the wearer of the orthopedic device during the opposite movement of the joint of the orthopedic device. During this process, the filling level of the compensation volume 24 remains unchanged.

FIG. 4 depicts the situation in which the first controllable valve 26 is open in accordance with the arrow 28. In a prosthetic knee, for example, this can occur during the swing phase, in which a flexion of the knee joint with as little resistance as possible is desired. The two throttle valves 16, 18 are opened, thereby enabling a fluid flow between the first cylinder chamber 6 and the second cylinder chamber 8 with as little resistance as possible. Due to the reduction in overall volume of the first cylinder chamber 6 and the second cylinder chamber 8, this causes the compensation volume 24 to be filled, which is indicated by the filling level 30.

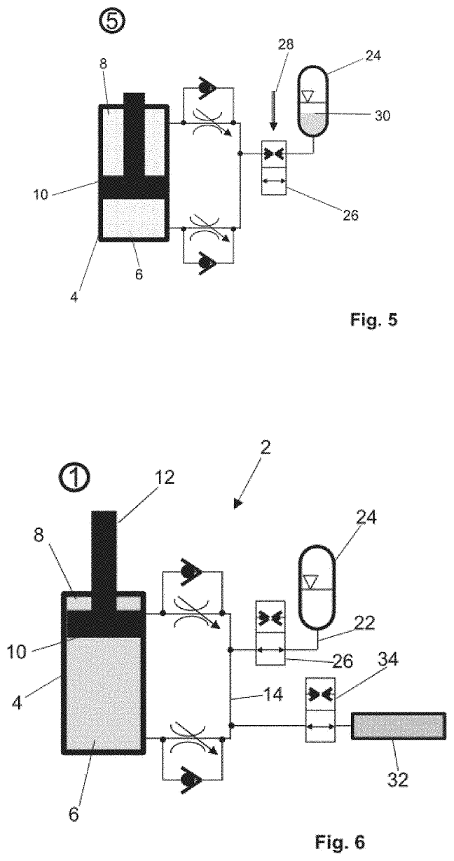

FIG. 5 depicts how the first controllable valve 26 is closed in this state in accordance with the arrow 28. The filling level 30 of the compensation volume 24 remains unchanged. In this state, a further displacement of the piston 10 inside the cylinder 4 leads to a change in the overall volume of the first cylinder chamber 6 and the second cylinder chamber 8, so that potential energy can be stored in the fluid, for example the silicone oil, said energy being released again once the force that produces it disappears.

FIGS. 6 to 10 depict a further embodiment of an energy storage device 2. It also features the cylinder 4 with the first cylinder chamber 6, second cylinder chamber 8, piston 10 and piston rod 12. The compensation volume 24 is connected via the fluid connection 22 to the fluid line 14 such that it can be switched via the first controllable valve 26, the previously known valves being located in said fluid line. In addition to the embodiment from FIGS. 1 to 5, the energy storage device 2 according to FIGS. 6 to 10 has an additional volume 32 that can be connected to or disconnected from the fluid line 14 via a third controllable valve 34.

In FIG. 6 both the first controllable valve 26 and the third controllable valve 34 are open, so that both the compensation volume 24 and the additional volume 32 are connected to the fluid line 14 and therefore also to the first cylinder chamber 6 and the second cylinder chamber 8.

If such an energy storage device 2 is installed in a prosthetic knee, for example, the embodiment can render sitting down and in particular standing up later much easier for the wearer of the orthopedic device, i.e. the prosthetic knee in this case.

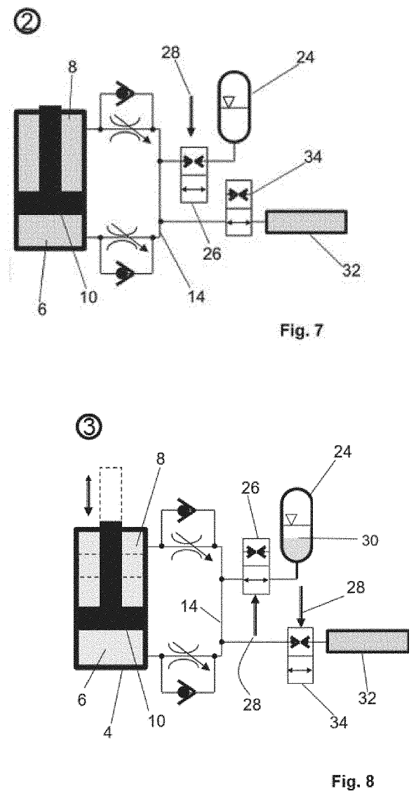

For sitting down itself, the switch arrangement shown in FIG. 7 is used. In accordance with the arrow 28, the switch valve 26 is closed, so that the compensation volume 24 is decoupled from the fluid line 14. The third controllable valve 34 remains open. When the piston 10 is lowered into the first cylinder chamber 6, as already shown in FIGS. 1 to 5, the overall volume of the first cylinder chamber 6 and the second cylinder chamber 8 is reduced, which of course is not changed by the additional volume 32 still connected to the fluid line 14. The overall volume available to the operating medium decreases, so that the fluid, for example the silicone oil, is compressed. In this state, potential energy is therefore stored in the energy storage device 2.

After sitting down, the switch arrangement shown in FIG. 8 is used. In accordance with the arrow 28, the first controllable valve 26 is opened, so that the compensation volume 24 is coupled with the fluid line 14. The third controllable valve 34 is also actuated and brought into the closed state, so that the additional volume 32 is decoupled from the rest of the system. It should be noted that preferably the third controllable valve 34 is actuated before the first controllable valve 26 in order to prevent a complete pressure equalization in the additional volume 32 as well. In both cylinder chambers 6, 8 the operating medium is under increased pressure following compression while sitting down, during which the piston 10 was lowered inside the cylinder 4. If the first controllable valve 26 is opened, this pressure can expand, wherein part of the fluid is pushed into the compensation volume 24, depicted by the filling level 30. If both throttle valves 16, 18 are now opened as far as possible, the opposing flow resistance is minimal, thereby enabling almost free movement of the knee. This is particularly desirable in the seated state.

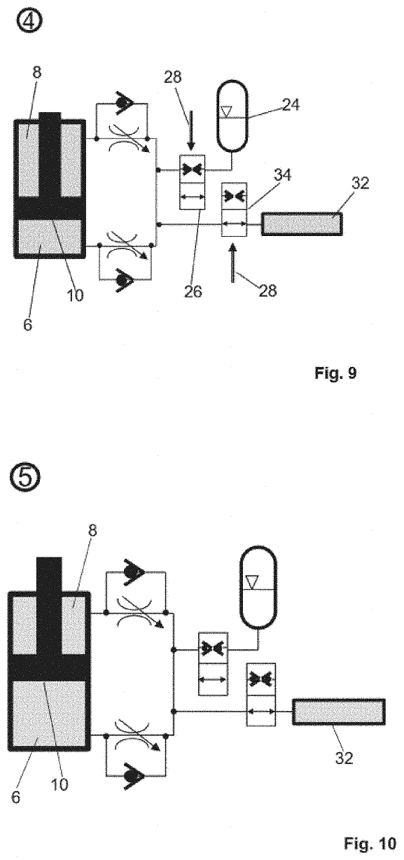

When standing up again, the switch arrangement shown in FIG. 9 is used. The two controllable valves 26, 34 are actuated in accordance with the arrows 28. However, the piston 10 is first brought back into the position that corresponds to a fully flexed knee, which was also achieved when sitting down. The first controllable valve 26 is consequently actuated and the compensation volume 24 disconnected from the rest of the fluid system. The third controllable valve 34 can then be actuated and brought into the open state, so that the additional volume 32 is re-connected to the rest of the fluid system. The highly pressurized fluid is still in said system, the fluid now ensuring a pressure equalization with the two cylinder chambers 6, 8 as well.

This now increased pressure provides an upward force on the piston 10, so that the piston 10 is pushed upwards out of the cylinder. This is shown in FIG. 10. The operating medium contained in the first cylinder chamber 6, the second cylinder chamber 8 and the additional volume 32 expands and releases its stored potential energy. As a result, the piston 10 is pushed upwards and the wearer of the orthopedic device, for example the prosthetic knee, is supported while standing up.

Of course, the arrangements can also be installed in other orthopedic devices, so that a displacement of the piston 10 inside the cylinder 4 does not correspond to a bending of the knee, but the movement of another joint.

### REFERENCE LIST

2 energy storage device
4 cylinder
6 first cylinder chamber
8 second cylinder chamber
10 piston
12 piston rod
14 fluid line
16 first throttle valve
18 second throttle valve
20 non-return valve
22 fluid connection
24 compensation volume
26 first controllable valve
28 arrow
30 filling level
32 additional volume
34 third controllable valve

1-12. (canceled)

13. An orthopedic device with an energy storage device that comprises at least one cylinder in which a first cylinder chamber, a second cylinder chamber, which is fluidically connected to the first cylinder chamber by at least one fluid line, and a piston, are located,
wherein the piston is arranged relative to the cylinder such that displacing the piston causes an operating medium, which is a fluid, to be conveyed through the at least one fluid line from one of the first or second cylinder chamber into the other of the first or second cylinder chamber, and
the energy storage device has at least one compensation volume, which is fluidically connected to the fluid line via a fluid connection, and a first controllable valve configured to open and close the fluid connection.

14. The orthopedic device according to claim 13, wherein the operating medium is a compressible fluid, preferably an oil, especially preferably a silicone oil.

15. The orthopedic device according to claim 13, wherein the operating medium is an oil.

16. The orthopedic device according to claim 13, wherein the operating medium is a silicone oil.

17. The orthopedic device according to claim 13, further comprising at least one second controllable valve in the fluid line configured to adjust a flow resistance of the fluid connection.

18. The orthopedic device according to claim 17, wherein the fluid connection is located between the first and second controllable valves in the fluid line.

19. The orthopedic device according to claim 13, wherein the energy storage device comprises at least one additional volume that is fluidically connected to at least one of the first cylinder chamber or the second cylinder chamber,

20. The orthopedic device according to claim 19, the energy storage device (2) having a third controllable valve (34) configured to open and close the connection.

21. The orthopedic device according to claim 19, wherein the energy storage device has multiple additional volumes and multiple third controllable valves configured to open and close the connections of the additional volumes to at least one of the first cylinder chamber or the second cylinder chamber

22. The orthopedic device according to claim 21, wherein the multiple third controllable valves are capable of opening and closing independently of each other.

23. The orthopedic device according to claim 21, wherein the multiple additional volumes are fluidically connected to each other in series.

24. The orthopedic device according to claim 21, wherein the multiple additional volumes are fluidcally connected to each other in parallel.

25. The orthopedic device according to claim 13, further comprising an electric control unit that is configured to control the controllable valves independently of each other.

26. The orthopedic device according to claim 13, wherein the piston is displaceable along a circular path.

27. The orthopedic device according to claim 13, wherein the device is a knee prosthesis or a knee orthosis.

28. The orthopedic device according to claim 13, wherein at least one of a diameter of a piston rod, a volume of the first cylinder chamber, a volume of the second cylinder chamber or a compression modulus of the operating medium are selected in such a way that a spring constant of at most 750 N/mm occurs when the fluid connection is closed.

29. The orthopedic device according to claim 28, wherein the spring constant is less than 600 N/mm.

30. The orthopedic device according to claim 28, wherein the spring constant is less than 400 N/mm.

31. The orthopedic device according to claim 28 wherein the spring constant is greater than 100 N/mm.

32. An energy storage device \ for an orthopedic device, the energy storing device comprising:
at least one cylinder,
a first cylinder chamber located in the at least one cylinder,
a second cylinder chamber located in the at least one cylinder, wherein the second cylinder chamber is fluidically connected to the first cylinder chamber by at least one fluid line,
a piston located in the at least one cylinder,
at least one compensation volume, which is fluidically connected to the fluid line via a fluid connection, and
a first controllable valve configured to open and close the fluid connection,
wherein the piston is arranged relative to the cylinder such that displacing the piston causes an operating medium, which is a fluid, to be conveyed through the at least one fluid line from one of the first or second cylinder chamber into the other of the first or second cylinder chamber.
